(19) United States
(12) Patent Application Publication
(10) Pub. No.: US 2005/0123565 A1
(43) Pub. Date: Jun. 9, 2005

(54) System and method for transdermal vaccine delivery

(76) Inventors: Janardhanan Subramony, Santa Clara, CA (US); Joseph B. Phipps, Sunnyvale, CA (US); Michel J.N. Cormier, Mountain View, CA (US); Georg Widera, Palo Alto, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/971,877

(22) Filed: Oct. 21, 2004

Related U.S. Application Data
(60) Provisional application No. 60/516,184, filed on Oct. 31, 2003.

(51) Int. Cl. A61N 1/30; A61K 39/02

(52) U.S. Cl. 424/234.1; 604/20

(57) ABSTRACT

A system and method for transdermally delivering a vaccine to a patient including an iontophoresis delivery device having a donor electrode, a counter electrode, and electric circuitry for supplying iontophoresis energy to the electrodes, and a non-electroactive microprojection member having a plurality of stratum corneum-piercing microprojections extending therefrom. The vaccine can be contained in a hydrogel formulation in an agent reservoir disposed proximate the donor electrode, in a biocompatible coating that is disposed on the microprojections or in both.

### CROSS-REFERENCE TO RELATED APPLICATIONS

This application claims the benefit of U.S. Provisional Application No. 60/516,184, filed Oct. 31, 2003.

### FIELD OF THE PRESENT INVENTION

The present invention relates generally to transdermal delivery systems and methods. More particularly, the invention relates to a percutaneous and intracellular vaccine delivery system and method.

### BACKGROUND OF THE INVENTION

Active agents (or drugs) are most conventionally administered either orally or by injection. Unfortunately, many active agents are completely ineffective or have radically reduced efficacy when orally administered since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure that sometimes results in poor patient compliance.

The word “transdermal” is used herein as a generic term referring to passage of an agent across the skin layers. The word “transdermal” refers to delivery of an agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis and electroporation) and ultrasound (e.g., phonophoresis).

While active agents do diffuse across both the stratum corneum and the epidermis, the rate of diffusion through the stratum corneum is often the limiting step. Many compounds, in order to achieve an effective dose, require higher delivery rates than can be achieved by simple passive transdermal diffusion.

Hence, in principle, transdermal delivery provides for a method of administering active agents that would otherwise need to be delivered orally or via hypodermic injection or intravenous infusion. Transdermal agent delivery offers improvements in these areas. Transdermal delivery, when compared to oral delivery, avoids the harsh environment of the digestive tract, bypasses gastrointestinal agent metabolism, reduces first-pass effects, and avoids the possible deactivation by digestive and liver enzymes. Likewise, the digestive tract is not subjected to the active agent during transdermal administration since many agents, such as aspirin, have an adverse effect on the digestive tract.

Transdermal delivery also offers advantages over the more invasive hypodermic or intravenous agent delivery options. Specifically, no significant cutting or penetration of the skin is necessary, such as cutting with a surgical knife or piercing the skin with a hypodermic needle. This minimizes the risk of infection and pain.

Transdermal delivery additionally offers significant advantages for vaccination, given the function of the skin as an immune organ. Pathogens entering the skin are confronted with a highly organized and diverse population of specialized cells capable of eliminating microorganisms through a variety of mechanisms. Epidermal Langerhans cells are potent antigen-presenting cells. Lymphocytes and dermal macrophages percolate throughout the dermis. Keratinocytes and Langerhans cells express or can be induced to generate a diverse array of immunologically active compounds. Collectively, these cells orchestrate a complex series of events that ultimately control both innate and specific immune responses.

It is further thought that non-replicating antigens (i.e., killed viruses, bacteria, an subunit vaccines) enter the endosomal pathway of antigen presenting cells. The antigens are processed and expressed on the cell surface in association with class II MHC molecules, leading to the activation of CD4⁺ T cells. Experimental evidence indicates that introduction of antigens exogenously induces little or no cell surface antigen expression associated with class I MHC, resulting in ineffective CD8⁺ T activation. Replicating vaccines, on the other hand (e.g., live, attenuated viruses such as polio and smallpox vaccines) lead to effective humoral and cellular immune responses and are considered the “gold standard” among vaccines. A similar broad immune response spectrum can be achieved by DNA vaccines.

In contrast, protein based vaccines, as subunit vaccines, and killed viral and bacterial vaccines do elicit predominantly a humoral response, as the original antigen presentation occurs via the class II MHC pathway. A method to enable the presentation of these vaccines also via the class I MHC pathway would be of great value, as it would widen the immune response spectrum.

Several reports have suggested that soluble protein antigens can be formulated with surfactants, leading to antigen presentation via the class I pathway and induce antigen-specific class I-restricted CTLs (Raychaudhuri et al 1992). Introduction of protein antigen by osmotic lysis of pinosomes has also been demonstrated to lead to a class I antigen-processing pathway (Moore, et al). Electroporation techniques have been typically used to introduce macromolecules into cells in vitro and in vivo, and particularly DNA-based therapeutics. Studies with plasmid DNA encoding target antigens have clearly demonstrated that the delivery efficiency can be significantly increased when electroporation is used. Proteins such as antibodies have been delivered into cells also using electroporation, demonstration functional inhibition of a intracellular target enzyme (Chakrabarti, et al).

Electroporation has been used to deliver biologics intracellularly in vivo and in vivo through various routes of administration, including transdermal. It is recognized that DNA vaccines can be delivered and expressed using this technology. Unfortunately, it is also known that electroporation in conscious patients is not practical because of the pain and muscle reaction associated with invasive electrodes and the strong electrical pulses involved.

Conversely, iontophoresis, which is being used to deliver pharmacological agents through mucosal and transdermal administration, is relatively non-invasive, well tolerated, and is being developed for use in conscious or ambulatory patients.

It would therefore be advantageous to employ iontophoresis for transdermal and intracellular delivery of vaccines. Unfortunately, iontophoretic delivery has limited capability for delivering high molecular weight compounds transdermally.

There have been many attempts to enhance transdermal flux by mechanically puncturing the skin prior to transdermal drug delivery. See, for example, U.S. Pat. Nos. 5,279,544, 5,250,023 and 3,964,482. In addition, U.S. Pat. Application No. 2002/0016562 teaches the use of iontophoresis in combination with a microprojection array.

There is, however, no published literature regarding in vivo intracellular iontophoresis delivery of protein-based vaccine molecules into skin antigen-presenting cells (APC) that leads to cellular loading of the protein epitopes onto class I MHC/HLA presentation molecules in addition to class II MHC/HLA presentation molecules. In particular, there is no mention of the use of a coated microprojection array in conjunction with iontophoresis to achieve the noted delivery.

There is also no published literature mentioning the use of a coated microprojection array in conjunction with iontophoresis to achieve in vivo delivery of a DNA vaccine intracellularly and subsequent cellular expression of the vaccine antigen encoded by the DNA vaccine and loading of the protein epitopes onto class I MHC/HLA presentation molecules in addition to class II MHC/HLA presentation molecules.

It is therefore an object of the present invention to provide a transdermal agent delivery system and method that substantially reduces or eliminates the aforementioned drawbacks and disadvantages associated with prior art agent delivery systems.

It is another object of the present invention to provide a system and method for transdermal vaccine delivery into skin antigen-presenting cells (“APC”).

It is another object of the present invention to provide a system and method for transdermal vaccine that employs an iontophoresis process to enhance the vaccine flux into the skin and into immunologically relevant skin cells.

### SUMMARY OF THE INVENTION

In accordance with the above objects and those that will be mentioned and will become apparent below, the system and method for transdermally delivering a vaccine in accordance with this invention comprises an iontophoresis device having a donor electrode, a counter electrode, electric circuitry for supplying iontophoresis energy to the electrodes, a formulation adapted for transdermal delivery containing the vaccine, and a non-electroactive microprojection member having a plurality of stratum corneum-piercing microprojections extending therefrom.

In one embodiment of the invention, the microprojection member has a microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials.

In a most preferred embodiment, the microprojection member is constructed out of a non-conductive material, such as a polymer. Alternatively, the microprojection member can be coated with a non-conductive material, such as Parylene®.

In one embodiment of the invention, the microprojection member is a separate component.

In an alternative embodiment, the microprojection member is disposed proximate the donor electrode of the iontophoresis device.

The vaccine can include viruses and bacteria, protein-based vaccines, polysaccharide-based vaccine, and nucleic acid-based vaccines.

In one embodiment of the invention, the vaccine is a protein-based vaccine. In such an embodiment, application of the iontophoresis energy to the electrodes preferably provides in vivo intracellular delivery of the protein-based vaccine, whereby delivery of the protein-based vaccine into skin-presenting cells leads to cellular loading of the protein-based vaccine epitopes onto class I MHC/HLA presentation molecules in addition to class II MHC/HLA presentation molecules in a subject.

In a further aspect, a cellular and humoral response is produced in the subject.

In another embodiment of the invention, the vaccine is a DNA vaccine. In such an embodiment, application of the iontophoresis energy to the electrodes preferably provides in vivo intracellular delivery of the DNA-based vaccine and subsequent cellular expression of the vaccine antigen encoded by the DNA vaccine and loading of the protein epitopes onto class I MHC/HLA presentation molecules in addition to class II MHC/HLA presentation molecules.

In an additional aspect, a cellular and humoral response is produced in the subject. Alternatively, only a cellular response is produced.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linke to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitdis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, lyme disease vaccine, rabies vaccine, measles vaccine, mumps vaccine, chicken pox vaccine, small pox vaccine, hepatitus vaccine, pertussis vaccine, and diptheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; sclavo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

In a preferred embodiment of the invention, the formulation comprises a biocompatible coating that is disposed on the microprojection member.

The coating formulations applied to the microprojection member to form solid coatings can comprise aqueous and non-aqueous formulations having at least one vaccine, which can be dissolved within a biocompatible carrier or suspended within the carrier.

In one embodiment of the invention, the coating formulations include at least one surfactant, which can be zwitterionic, amphoteric, cationic, anionic, or nonionic, comprises sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols such as laureth-4.

In one embodiment of the invention, the concentration of the surfactant is in the range of approximately 0.001-2 wt. % of the coating solution formulation.

In a further embodiment of the invention, the coating formulations include at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating.

In another embodiment, the coating formulations include a hydrophilic polymer selected from the following group: poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 0.01-20 wt. %, more preferably, in the range of approximately 0.03-10 wt. % of the coating formulation.

In another embodiment of the invention, the coating formulations include a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In a further embodiment, the coating formulations include a stabilizing agent, which can comprise, without limitation, a non-reducing sugar, a polysaccharide, a reducing sugar, or a DNase inhibitor.

In another embodiment, the coating formulations include a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

Preferably, the coating formulations have a viscosity less than approximately 500 centipoise and greater than 3 centipoise.

In one embodiment of the invention, the coating thickness is less than 25 microns, more preferably, less than 10 microns as measured from the microprojection surface.

In other embodiments of the invention, the formulation comprises a hydrogel which can be incorporated into a gel pack. Preferably, the system further comprises an agent reservoir disposed adjacent the donor electrode that is adapted to contain the hydrogel formulation.

Correspondingly, in certain embodiments of the invention, the hydrogel formulations contain at least one vaccine or immunologically active agent. Preferably, the agent comprises one of the aforementioned vaccines, including, without limitation, viruses and bacteria, protein-based vaccines, polysaccharide-based vaccine, and nucleic acid-based vaccines.

The hydrogel formulation(s) contained in the donor reservoir preferably comprise water-based hydrogels having macromolecular polymeric networks.

In a preferred embodiment of the invention, the polymer network comprises, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

The hydrogel formulations preferably include one surfactant, which can be zwitterionic, amphoteric, cationic, anionic, or nonionic.

In one embodiment of the invention, the surfactant can comprise sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives such as sorbitan laurate, and alkoxylated alcohols such as laureth-4.

In another embodiment, the hydrogel formulations include polymeric materials or polymers having amphiphilic properties, which can comprise, without limitation, cellulose derivatives, such as hydroxyethylcellulose (HEC), hydroxypropylethylcellulose (HPMC), hydroxypropycellulose (HPC), methylcellulose (MC), hydroxyethylmethyl-cellulose (HEMC), or ethylhydroxyethylcellulose (EHEC), as well as pluronics.

In a further embodiment of the invention, the hydrogel formulations contain at least one pathway patency modulator, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, and EDTA.

In yet another embodiment of the invention, the hydrogel formulations include at least one vasoconstrictor, which can comprise, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

According to the invention, the vaccine to be delivered can be contained in the hydrogel formulation disposed in a gel pack reservoir, contained in a biocompatible coating that is disposed on the microprojection member or contained in both the hydrogel formulation and the biocompatible coating. Furthermore, embodiments that comprise the vaccine in a coating can also employ a hydrogel reservoir to hydrate and dissolve the coating.

In accordance with one embodiment of the methods of the invention, the vaccine(s) (contained in the hydrogel formulation, disposed in the agent reservoir, contained in the biocompatible coating on the microprojection member or both) is delivered to the patient via the iontophoresis device as follows: the system discussed above is placed in intimate contact with the patient skin, wherein the microprojections pierce the stratum corneum, current is applied to the electrodes and the vaccine is delivered.

In one embodiment, the microprojection member is integral with the electrodes, and thus current is applied prior to removal of the microprojection member.

In accordance with a further preferred embodiment, the coated microprojection member is initially applied to the patient's skin, preferably via an impact applicator, the iontophoresis device is then applied on the skin, whereby the electrode assembly contacts the applied microprojection member. Also preferably, the applicator is capable of applying the microprojection member in such a manner that said microprojection member strikes the stratum corneum of a patient with a power of at least 0.05 joules per cm² of microprojection member in 10 milliseconds or less.

In an alternative embodiment, after application and removal of the coated microprojection member, the iontophoresis device is then placed on the patient's skin proximate the pre-treated area.

In one embodiment of the invention, after the iontophoresis device is placed on the patient's skin, a current in the range of approximately 50 μA-20 mA is applied over a time period that ranges from 10 seconds to 1 day.

In an alternative embodiment, after the iontophoresis device is placed on the patient's skin, a voltage in the range of approximately 0.5 V-20 V is applied over a time period that ranges from 10 seconds to 1 day.

In one embodiment of the invention, after the iontophoresis device is placed on the patient's skin, the target amperage or voltage is achieved by a slow ramping up of the applied electric condition.

In an alternative embodiment, starting from the target amperage or voltage, the electrical conditions are ramped down over time.

In another alternative embodiment, consecutive pulses lasting from 1 second to 12 hours, using the above electrical conditions are applied during the total duration of iontophoresis.

In methods of the invention wherein the vaccine is a protein-based vaccine, application of the iontophoresis energy to the electrodes preferably provides in vivo intracellular delivery of the protein-based vaccine, whereby delivery of the protein-based vaccine into skin-presenting cells leads to cellular loading of the vaccine epitopes onto class I MHC/HLA presentation molecules in addition to class II MHC/HLA presentation molecules in a subject. Also preferably, a cellular and humoral response is produced in said subject.

In methods of the invention wherein the vaccine is a DNA vaccine, application of the iontophoresis energy to the electrodes preferably provides in vivo intracellular delivery of the DNA-based vaccine, whereby delivery of the DNA-based vaccine into skin-presenting cells leads to cellular expression of the vaccine antigen encoded by the DNA vaccine and loading of the vaccine epitopes onto class I MHC/HLA presentation molecules in addition to class II MHC/HLA presentation molecules in a subject. Also preferably, a cellular and humoral response is produced in said subject. Alternatively, only a cellular response is produced.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of one embodiment of an iontophoresis device for transdermally delivering a vaccine, according to the invention;

FIG. 2 is a schematic illustration of a further embodiment of an iontophoresis device for transdermally delivering a vaccine, according to the invention;

FIG. 3 is a perspective view of a portion of one example of a microprojection array;

FIG. 4 is a perspective view of the microprojection array shown in FIG. 3 having a coating deposited on the microprojections, according to the invention;

FIG. 4A is a cross-sectional view of a single microprojection taken along line 2A-2A in FIG. 4, according to the invention;

FIG. 5 is a side sectional view of a microprojection array having an adhesive backing;

FIG. 6 is a side sectional view of a retainer having a microprojection member disposed therein; and

FIG. 7 is a perspective view of the retainer shown in FIG. 6.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin.

The term “transdermal flux”, as used herein, means the rate of transdermal delivery.

The term “vaccine”, as used herein, refers to a composition of matter or mixture containing an immunologically active agent or an agent, such as an antigen, which is capable of triggering a beneficial immune response when administered in an immunologically effective amount. Examples of such agents include, without limitation, viruses and bacteria, protein-based vaccines, polysaccharide-based vaccine, and nucleic acid-based vaccines.

Particularly with regard to protein-based vaccines and DNA vaccines, iontophoresis preferably provides in vivo intracellular delivery of the vaccine. In the case of protein-based vaccines, this delivery into skin-presenting cells leads to cellular loading of the protein-based vaccine epitopes onto class I MHC/HLA presentation molecules in addition to class II MHC/HLA presentation molecules in a subject. Preferably, a cellular and humoral response is produced.

With respect to DNA vaccines, delivery of the DNA-based vaccine into skin-presenting cells leads to cellular expression of the vaccine antigen encoded by the DNA vaccine and loading of the vaccine epitopes onto class I MHC/HLA presentation molecules in addition to class II MHC/HLA presentation molecules in a subject. Also preferably, a cellular and humoral response in produced in the subject. Alternatively, only a cellular response is produced.

Suitable antigenic agents that can be used in the present invention include, without limitation, antigens in the form of proteins, polysaccharide conjugates, oligosaccharides, and lipoproteins. These subunit vaccines in include Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxine subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitidis (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitidis, pseudomonas aeruginosa, streptococcus pneumoniae, treponema pallidum, and vibrio cholerae, and mixtures thereof.

A number of commercially available vaccines, which contain antigenic agents, also have utility with the present invention including, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids that can be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; sclavo peptide: VQGEESNDK.HCl (IL-11163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

The noted vaccines can also be in various forms, such as free bases, acids, charged or uncharged molecules, components of molecular complexes or pharmaceutically acceptable salts. Further, simple derivatives of the active agents (such as ethers, esters, amides, etc.), which are easily hydrolyzed at body pH, enzymes, etc., can be employed.

As will be appreciated by one having ordinary skill in the art, with few exceptions, alum-adjuvanted vaccine formulations typically lose potency upon freezing and drying. To preserve the potency and/or immunogenicity of the alum-adsorbed vaccine formulations of the invention, the noted formulations can be further processed as disclosed in Provisional Application No. ______ [Attorney Docket No. ALZ5156PSP1, filed Sep. 28, 2004]; which is expressly incorporated by reference herein in its entirety.

It is to be understood that more than one vaccine may be incorporated into the agent source, reservoirs, and/or coatings of this invention, and that the use of the term “active agent” in no way excludes the use of two or more such active agents or drugs.

The term “biologically effective amount” or “biologically effective rate” shall be used when the vaccine is an immunologically active agent and refers to the amount or rate of the immunologically active agent needed to stimulate or initiate the desired immunologic, often beneficial result. The amount of the immunologically active agent employed in the hydrogel formulations and coatings of the invention will be that amount necessary to deliver an amount of the active agent needed to achieve the desired immunological result. In practice, this will vary widely depending upon the particular immunologically active agent being delivered, the site of delivery, and the dissolution and release kinetics for delivery of the active agent into skin tissues.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

In one embodiment of the invention, the piercing elements have a projection length less than 1000 microns. In a further embodiment, the piercing elements have a projection length of less than 500 microns, more preferably, less than 250 microns. The microprojections typically have a width and thickness of about 5 to 50 microns. The microprojections may be formed in different shapes, such as needles, hollow needles, blades, pins, punches, and combinations thereof.

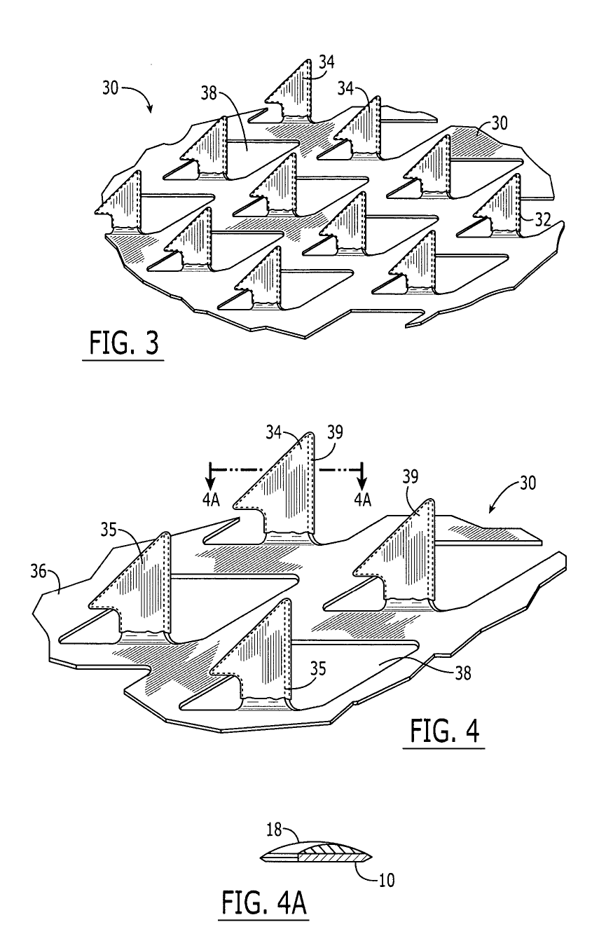

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 3. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “iontophoresis”, as used herein, refers generally to the delivery of a therapeutic agent (charged, uncharged, or mixtures thereof) through a body surface (such as skin, mucous membrane, or nails) wherein the delivery is at least partially induced or aided by the application of an electric potential. As is known in the art, iontophoresis, an electrotransport process, involves the electrically induced transport of charged ions.

Electroosmosis, another type of electrotransport process involved in the transdermal transport of uncharged or neutrally charged molecules (e.g., transdermal sampling of glucose), involves the movement of a solvent with the agent through a membrane under the influence of an electric field.

In many instances, more than one of the noted processes may be occurring simultaneously to different extents. Accordingly, the term “iontophoresis” is given herein its broadest possible interpretation, to include the electrically induced or enhanced transport of at least one charged or uncharged agent, or mixtures thereof, regardless of the specific mechanism(s) by which the agent is actually being transported.

In typical transdermal iontophoresis system a low constant current, ranging from micro-Amps to several milli-Amps, is applied for prolonged periods of time ranging from minutes to days. Alternatively, low constant voltage, ranging from milli volts to several volts is applied for prolonged periods of time ranging from minutes to days. The target amperage or voltage may also be achieved by a slow ramping up of the applied electric condition. Alternatively, starting from the target amperage or voltage, the electrical conditions may also be ramped down over time. Alternatively, consecutive pulses using the above electrical conditions are applied during the total duration of iontophoresis. Collectively, the above electrical conditions are referred to herein as “iontophoresis energy”. The above conditions are different from the electrical conditions as applied in the field of electroporation and do not result in measurable pore formation through cell membrane.

The term “electroporation”, as used herein, generally recognizes that exposing cells to strong electric fields for brief periods of time can temporarily destabilize the cell membranes. This effect has been described as a dielectric breakdown due to an induced transmembrane potential, and may also be referred to as “electropermeabilization.” Preferably, the permeabilized state of the cell membrane is transitory. Typically, cells remain in a destabilized state on the order of minutes after electrical treatment ceases. Electrical fields for poration are commonly generated by capacitor discharge power units using pulses of very short (micro to millisecond) time course and field strength greater than 50 V/cm. Square wave and radio frequency pulses have also been used for cell electroporation.

As indicated above, the present invention comprises a system and method for transdermally delivering a vaccine to a patient. The system generally includes an iontophoresis delivery device having a donor electrode, a counter electrode, and electric circuitry for supplying iontophoresis energy to the electrodes, and a non-electroactive microprojection member having a plurality of stratum corneum-piercing microprojections extending therefrom.

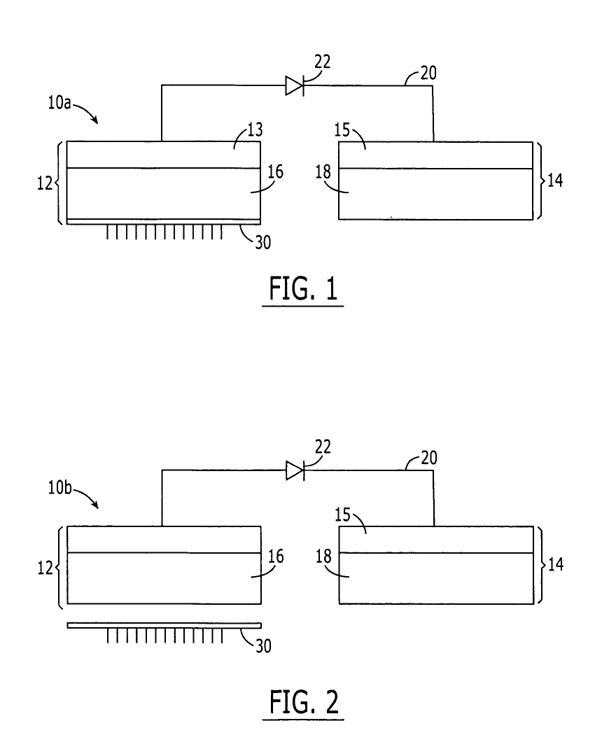

Referring now to FIGS. 1 and 2, there are shown schematic illustrations of exemplary iontophoresis devices that can be used in accordance with the present invention. Referring first to FIG. 1, the iontophoresis device 10a generally includes a donor electrode assembly 12 and a counter electrode assembly 14. These designations of the electrode assemblies 12, 14 are not critical and may be reverse in any particular device or in operation of the device 10 shown.

The electrode assemblies can further be separate units, as shown in FIG. 1, or an integral unit having an electrical insulator therebetween.

The iontophoresis device 10a further includes an electric circuit 20 that is in communication with the electrode assemblies 12 and 14 and a suitable power source 22, such as a battery.

Referring back to FIG. 1, the electrode assembly 12 includes a donor electrode 13 preferably disposed adjacent to an agent reservoir 16. The agent reservoir 16 is adapted to receive the agent formulation (e.g., hydrogel formulation) therein. An ionic exchange membrane (not shown) can be optionally intercalated between the agent reservoir 16 and the donor electrode 13 in order to minimize ionic competition. Additionally, an electrolyte hydrogel (not shown) can be optionally intercalated between the ionic exchange membrane and the donor electrode 13.

As illustrated in FIG. 1, the electrode assembly 14 includes a counter electrode 15 preferably disposed adjacent to a return reservoir 18. The return reservoir 18 is adapted to receive a suitable electrolyte, such as a saline hydrogel, therein.

The donor and counter electrodes are preferably composed of electrically conductive material, such as a metal. For example, the electrodes can be formed from a metal foil, a metal screen, on metal deposited or painted on a suitable backing or by calendaring, film evaporation, or by mixing the electrically conductive material in a polymer binder matrix. Examples of suitable electrically conductive materials include, without limitation, carbon, graphite, silver, zinc, aluminum, platinum, stainless steel, gold and titanium. For example, as noted above, the anodic electrode can be composed of silver, which is also electrochemically oxidizable. The cathodic electrode can be composed of carbon and electrochemically reducible silver chloride.

Silver is preferred over other metals because of its relatively low toxicity to mammals. Silver chloride is preferred because the electrochemical reduction reaction occurring at the cathode (AgCl+c.sup.−AG+Cl.sup.−) produces chloride ions, which are prevalent in, and non-toxic to, most mammals.

The donor and counter electrodes are directly connected to the electrical circuit 20 and are defined herein as “electroactive”.

The iontophoresis device 10a further includes a microprojection member 30 that, in a preferred embodiment, is disposed proximate the electrode assembly 12. As discussed in detail below, the microprojection member 30 includes a plurality of microprojections 34 (or array thereof) that are adapted to pierce the stratum corneum when applied to a patient (see FIG. 3).

Referring now to FIG. 2, there is shown a further iontophoresis device 10b that can be employed within the scope of the present invention. As illustrated in FIG. 2, the device 10b is essentially the same as the device 10a shown in FIG. 1, with the exception that the microprojection member 30 is a separate component.

Generally, the combined skin-contacting area of electrode assemblies 12, 14 can range from about 1 cm² to about 200 cm², but typically will range from about 5 cm² to about 50 cm².

According to the invention, the iontophoresis device 10b can be adhered to the skin by means of an optional ion-conducting adhesive layer. Alternatively, or in conjunction, the microprojections 34 can be configured as barbs to anchor the device to the skin.

The device 10a or 10b also preferably includes a strippable release liner that is removed just prior to application of the device to the skin. Alternatively, the device 10a or 10b can be adhered to the skin by means of an adhesive overlay of the type that is conventionally used in transdermal drug delivery devices.

Referring now to FIG. 3, there is shown one embodiment of a microprojection member 30 for use with the present invention. As illustrated in FIG. 3, the microprojection member 30 includes a microprojection array 32 having a plurality of microprojections 34. The microprojections 34 preferably extend at substantially a 90° angle from the sheet 36, which in the noted embodiment includes openings 38.

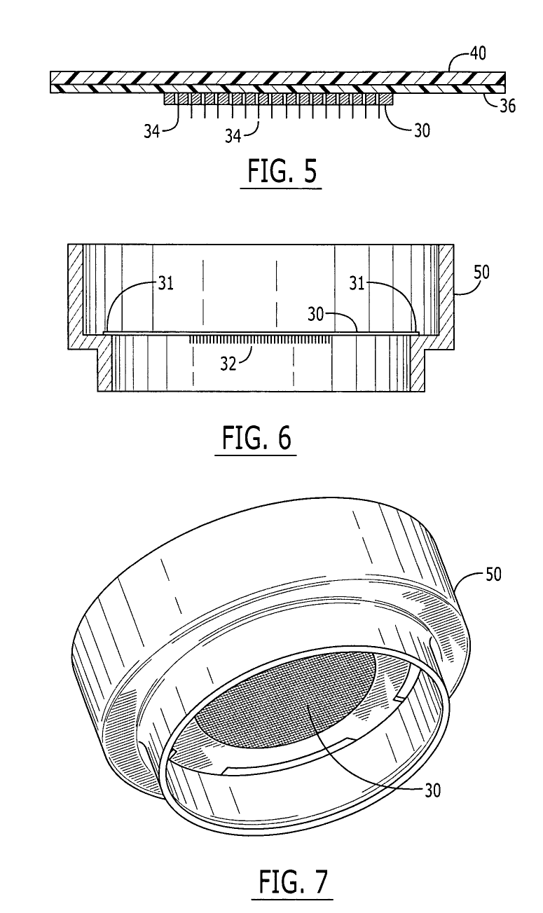

According to the invention, the sheet 36 may be incorporated into a delivery patch, including a backing 40 for the sheet 36, and may additionally include adhesive 16 for adhering the patch to the skin (see FIG. 5). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width and thickness of about 5 to 50 microns.

The microprojection member 30 can be manufactured from various metals, such as stainless steel, titanium, nickel titanium alloys, or similar biocompatible materials. Preferably, the microprojection member 30 is manufactured out of titanium.

According to the invention, the microprojection member 30 can also be constructed out of a non-conductive material, such as a polymer. Alternatively, the microprojection member can be coated with a non-conductive material, such as Parylene®.

According to the invention, the microprojection member 30 is preferably non-electroactive (i.e., is separated from the electrode 13 by an electrolyte).

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the biologically active agent (i.e., vaccine) to be delivered can be contained in the hydrogel formulation disposed in the agent reservoir 16, contained in a biocompatible coating that is disposed on the microprojection member 30 or contained in both the hydrogel formulation and the biocompatible coating.

Referring now to FIG. 4, there is shown a microprojection member 30 having microprojections 34 that include a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety.

As discussed in detail in the noted application, the disclosed roller coating method provides a smooth coating that is not easily dislodged from the microprojections 34 during skin piercing. The smooth cross-section of the microprojection tip coating is further illustrated in FIG. 2A.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

Another coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

As indicated, according to one embodiment of the invention, the coating formulations applied to the microprojection member 30 to form solid coatings can comprise aqueous and non-aqueous formulations having at least one biologically active agent, more preferably, a vaccine. According to the invention, the vaccine can be dissolved within a biocompatible carrier or suspended within the carrier.

According to the invention, the coating formulations preferably include at least one wetting agent. As is well known in the art, wetting agents can generally be described as amphiphilic molecules. When a solution containing the wetting agent is applied to a hydrophobic substrate, the hydrophobic groups of the molecule bind to the hydrophobic substrate, while the hydrophilic portion of the molecule stays in contact with water. As a result, the hydrophobic surface of the substrate is not coated with hydrophobic groups of the wetting agent, making it susceptible to wetting by the solvent. Wetting agents include surfactants as well as polymers presenting amphiphillic properties.

In one embodiment of the invention, the coating formulations include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

Preferably, the concentration of the surfactant is in the range of approximately 0.001-2 wt. % of the coating solution formulation.

In a further embodiment of the invention, the coating formulations include at least one polymeric material or polymer that has amphiphilic properties. Examples of the noted polymers include, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

As will be appreciated by one having ordinary skill in the art, the noted wetting agents can be used separately or in combinations.

According to the invention, the coating formulations can further include a hydrophilic polymer. Preferably the hydrophilic polymer is selected from the following group: poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

According to the invention, the coating formulations can further include a biocompatible carrier, such as those disclosed in Co-Pending U.S. application Ser. No. 10/127,108, which is incorporated by reference herein in its entirety. Examples of suitable biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation. Even more preferably, the concentration of the wetting agent is in the range of approximately 10-40 wt. % of the coating formulation.

According to the invention, the coating formulations can further include a stabilizing agent, such as those disclosed in Co-Pending U.S. Application No. 60/514,533, which is incorporated by reference herein in its entirety. Examples of suitable stabilizing agents include, without limitation, a non-reducing sugar, a polysaccharide, a reducing sugar, or a DNase inhibitor.

The coatings of the invention can further include a vasoconstrictor such as those disclosed in Co-Pending U.S. application Ser. Nos. 10/674,626 and 60/514,433, which are incorporated by reference herein in their entirety. As set forth in the noted Co-Pending Applications, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride), and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

According to the invention, the coating formulations can also include a non-aqueous solvent, such as ethanol, propylene glycol, polyethylene glycol and the like, dyes, pigments, inert fillers, permeation enhancers, excipients, and other conventional components of pharmaceutical products or transdermal devices known in the art.

Other known formulation additives can also be added to the coating formulations as long as they do not adversely affect the necessary solubility and viscosity characteristics of the coating formulation and the physical integrity of the dried coating.

Preferably, the coating formulations have a viscosity less than approximately 500 centipoise and greater than 3 centipoise in order to effectively coat each microprojection 10. More preferably, the coating formulations have a viscosity in the range of approximately 3-200 centipoise.

According to the invention, the desired coating thickness is dependent upon the density of the microprojections per unit area of the sheet and the viscosity and concentration of the coating composition as well as the coating method chosen. Preferably, the coating thickness is less than 50 microns.

In one embodiment, the coating thickness is less than 25 microns, more preferably, less than 10 microns as measured from the microprojection surface. Even more preferably, the coating thickness is in the range of approximately 1 to 10 microns.

In all cases, after a coating has been applied, the coating formulation is dried onto the microprojections 10 by various means. In a preferred embodiment of the invention, the coated member 5 is dried in ambient room conditions. However, various temperatures and humidity levels can be used to dry the coating formulation onto the microprojections. Additionally, the coated member 5 can be heated, lyophilized, freeze dried or similar techniques used to remove the water from the coating.

Referring now to FIGS. 6 and 7, for storage and application (in accordance with one embodiment of the invention), the microprojection member 30 is preferably suspended in a retainer ring 50 by adhesive tabs 31, as described in detail in Co-Pending U.S. application Ser. No. 09/976,762 (Pub. No. 2002/0091357), which is incorporated by reference herein in its entirety.

After placement of the microprojection member 30 in the retainer ring 50, the microprojection member 30 is applied to the patient's skin. Preferably, the microprojection member 30 is applied to the skin using an impact applicator, such as disclosed in Co-Pending U.S. application Ser. No. 09/976,798, which is incorporated by reference herein in its entirety.

In other aspects of the invention, the vaccine is contained in a hydrogel formulation. Preferably, the hydrogel formulation(s) contained in the donor reservoir 12 comprise water-based hydrogels, such as the hydrogel formulations disclosed in Co-Pending Application No. 60/514,433, which is incorporated by reference herein in its entirety.

As is well known in the art, hydrogels are macromolecular polymeric networks that are swollen in water. Examples of suitable polymeric networks include, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. These polymers can be obtained in various grades presenting different average molecular weights and therefore exhibit different rheological properties.

According to the invention, the hydrogel formulations also include one surfactant (i.e., wetting agent). According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives such as sorbitan laurate, and alkoxylated alcohols such as laureth-4. Most preferred surfactants include Tween 20, Tween 80, and SDS.

Preferably, the hydrogel formulations further include polymeric materials or polymers having amphiphilic properties. Examples of the noted polymers include, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

Preferably, the concentration of the surfactant is comprised between 0.001% and 2 wt. % of the hydrogel formulation. The concentration of the polymer that exhibits amphiphilic properties is preferably in the range of approximately 0.5-40 wt. % of the hydrogel formulation.

As indicated, according to at least one additional embodiment of the invention, the invention, the hydrogel formulations contain at least one biologically active agent, more preferably, a vaccine. Preferably, the vaccine comprises one of the aforementioned vaccines, including, without limitation, viruses and bacteria, protein-based vaccines, polysaccharide-based vaccine, and nucleic acid-based vaccines.

In a further embodiment of the invention, the hydrogel formulations contain at least one pathway patency modulator, such as those disclosed in Co-Pending U.S. application Ser. No. 09/950,436, which is incorporated by reference herein in its entirety. Suitable pathway patency modulators include, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, and EDTA.

According to the invention, the hydrogel formulations can also include a non-aqueous solvent, such as ethanol, isopropanol, propylene glycol, polyethylene glycol and the like, dyes, pigments, inert fillers, permeation enhancers, excipients, and other conventional components of pharmaceutical products or transdermal devices known in the art.

The hydrogel formulations can further include at least one vasoconstrictor. Suitable vasoconstrictors similarly include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

In accordance with one embodiment of the invention, the vaccine(s) (contained in the hydrogel formulation, disposed in the agent reservoir 16 or contained in the biocompatible coating on the microprojection member 30 or both) is delivered to the patient via an iontophoresis device, such as illustrated in FIG. 1, as follows: the device (e.g., 10a) is placed in intimate contact with the patient skin, wherein the microprojections 34 pierce the stratum corneum. Various sites on the human body may be selected depending upon the physician's or the patient's preference, the agent delivery regimen or other factors, such as cosmetic.

In accordance with a further preferred embodiment, the microprojection member 30 is initially applied to the patient's skin via an impact applicator or actuator, such as that disclosed in Co-Pending U.S. application Ser. No. 09/976,798, which is incorporated by reference herein in its entirety. After application of the microprojection member, as described in Co-Pending Application No. 60/514,433, the iontophoresis device 10a is applied on the skin, whereby the electrode assembly 12 contacts the microprojection member 30.

Alternatively, after application and removal of the microprojection member, as described in Co-Pending Application No. 60/514,387, the iontophoresis device 10b is then placed on the patient's skin proximate the pre-treated area.

According to the invention, after the device (10a or 10b) is placed on the patient's skin, a current in the range of 50 μA-20 mA is applied over a time period that ranges from 10 seconds to 1 day.

Alternatively, after the device is placed on the patient's skin, a voltage in the range of 0.5 V-20 V is applied over a time period that ranges from 10 seconds to 1 day.

According to the invention, after the device is placed on the patient's skin, the target amperage or voltage can also be achieved by a slow ramping up of the applied electric condition.

Alternatively, starting from the target amperage or voltage, the electrical conditions can also be ramped down over time.

Alternatively, consecutive pulses lasting from 1 second to 12 hours using the above electrical conditions are applied during the total duration of iontophoresis.

### EXAMPLES

The following example is given to enable those skilled in the art to more clearly understand and practice the present invention. It should not be considered as limiting the scope the invention but merely as being illustrated as representative thereof.

### Example 1

This experiment studied the effect of the mode of delivery of DNA and the effect of iontophoresis on gene expression of the marker gene encoded by the delivered DNA. A microprojection member combined with an iontophoresis device was used to increase intracellular delivery of DNA and gene expression in the hairless guinea pig (HGP). Six groups using microprojection array delivery, in addition to one DNA delivery group receiving DNA by intra-dermal injection using conventional hypodermic needles, and one negative control group were studied. Application of electroporation pulses through electroactive needle electrodes inserted into the skin is known to increase gene expression and was included in this experiment as a positive control.

Group 1: DNA delivery by coated microprojection array without any iontophoresis or electroporation.

Group 2: DNA delivery by coated microprojection array followed by electroporation applied through a separate electroactive 2×6 needle array electrode as a positive control.

Group 3: DNA delivery by coated microprojection array followed by cathodic iontophoresis using a donor electrode assembly containing a HEC gel after removal of the microprojection array.

Group 3A: DNA delivery by coated microprojection array followed by cathodic iontophoresis using a donor electrode assembly containing a DNA/HEC gel after removal of the microprojection array.

Group 4: DNA delivery by coated microprojection array followed by cathodic iontophoresis using a donor electrode assembly containing a HEC gel with the non-electroactive microprojection member left in the skin.

Group 4A: DNA delivery by microprojection array followed by cathodic iontophoresis using a donor electrode assembly containing a DNA/HEC gel with the non-electrocative microprojection member left in the skin.

Group 5: DNA delivery by intra-dermal injection.

Group 6: untreated skin.

### Materials and Methods

Two different microprojection arrays were used. Both arrays comprised titanium microprojections bent at an angle of approximately 90° to the plane of the sheet and an area of approximately 2 cm². The first array (1035) had a microprojection density of 657 microprojections/cm² and each microprojection had a length of 225 microns. The second array (1066) had a microprojection density of 140 microprojections/cm² and each microprojection had a length of 600 microns.

Both arrays were dry coated with a Green Fluorescent Protein (GFP) expression plasmid-40 μg DNA per array for array 1066 and 60 μg DNA per array for 1035. Two animal groups from Groups 2, 3, 3A, 4 and 4A received array 1035 and two animals from Groups 1, 2, 3, 3A, 4 and 4A received array 1066.

For Groups 1 and 2, the system comprised an adhesive backing (diameter 2.6 cm) with a 2 cm² microprojection array adhered in the middle. For Groups 3 and 3A, the system comprised an adhesive ring adhered to an adhesive backing ring (diameter 2.6 cm) with a 2 cm microprojection array adhered in the middle. For Groups 4 and 4A, the system comprised an adhesive backing ring (diameter 2.6 cm) with a 2 cm² microprojection array adhered in the middle. For Groups 3, 3A, 4 and 4A, the iontophoresis gels used for the anode and cathode assemblies not containing DNA were 350 μl of aqueous 0.15 M NaCl in 2% HEC (NATROSOL® 250 HHX PHARM, HERCULES Int. Lim., Netherlands, determined molecular weight: Mw 1890000, Mn 1050000). For the cathodes with DNA in the gel, the donor electrode assemblies consisted of 350 μl 20 mM NaCl, 2% aqueous HEC gel proximate to the cathode and a 175 μl, 3.6 mg/ml DNA, 25 mM Gly-His, 0.2% Tween 20, 1.5% aqueous HEC gel proximate to the skin. The two gels were separated by a cationic exchange membrane (Nafion®).

The conditions used for the conventional needle electroporation (EP) electrodes were 4 EP pulses, 100V/cm, 40 msec., 2 Hz., delivered by a 2×6 needle array electrode (6NA, Cytopulse) inserted into the skin at the microprojection array delivery site. The distance between the positive and negative needle row was 6 mm and the length of the needles was 4 mm. The pulse generator used was a BioRad GenePulser Xcell™.

The iontophoresis (IO) conditions used for this example were a 4 mA setting for a total of 60 mA×minutes (15 minutes treatment time). Anode and cathode electrode assemblies were assembled immediately prior to application to skin by dispensing the indicated amounts of formulated HEC gels into the electrode assembly reservoirs. The distance (center) between anode and cathode was 3 cm. An iontophoresis power supply, DOMED phoresor II, Model No. PM100, was used.

Delivery of the DNA to the skin of HGPs was as follows. Coated microprojection arrays were applied to the flank of the anesthetized HGPs using an impact applicator. For Groups 1 and 2, 1 minute after array application, the microprojection array adhered to the adhesive backing was removed. For Group 2, immediately following removal of the microprojection arrays, the 6NA was inserted into the skin to the full length of the needles. For Groups 3 and 3A, 1 min after array application, the microprojection array adhered to the adhesive backing ring was removed, leaving the adhesive ring in contact with the skin. For Groups 3, 3A, 4 and 4A, 1 min following microprojection application, the donor electrode assembly was applied to the adhesive ring (Groups 3 and 3A) or to the adhesive backing ring of the microprojection array (Groups 4 and 4A). The electrical condition (EP or IO) was applied immediately following DNA delivery by microprojection array, while all animals remained under anesthesia.

The configuration of the microprojection array system and components thereof are described in detail in U.S. Pat. Application No. 2002/0128599 and U.S. Provisional Application Nos. 60/514,433 and 60/514,387. The impact applicator is described in detail in U.S. Pat. Application No. 2002/0123675.

Intracellular uptake of plasmid DNA after microprojection DNA delivery was determined by measuring gene expression of the encoded GFP protein on the mRNA level by reverse transcriptase polymerase chain reaction (rtPCR). One day (24 hrs.) after DNA delivery, the animals were sacrificed and 8 mm skin biopsies were obtained from the center of all treatment sites, intra dermal injection sites, and untreated skin sites. Biopsies were weighed, homogenized by mincing and short sonication. RNA was extracted using the Stratagen RNA extraction Kit (Absolutely RNA™ RT-PCR Miniprep Kit (Stratagene 400800) according to the manufacturer's protocol, and first strand cDNAs were generated using the ProSTAR First strand RT-PCR kit (Stratagene Cat# 200420). rtPCR reactions were performed using an Invitrogen Kit: PCR Supermix (Invitrogen 10572014).

PCR conditions for this example were as follows. The primers used included an Intron RT 5′ primer-5′CCG GGA ACG GTG CAT TGG AA 3′ [SEQ. ID NO. 1] and a 3′ p2243 primer-5′ TGCTTGGACTGGGCCATGGT 3′ [SEQ. ID NO. 2]. The fragments provided were 958 bp (plasmid) or 131 bp (message). 2 μl primers were used with 5 μg total starting RNA in a 50 μl reaction. The PCR reaction conditions were 95° C. for 5 min, 40 cycles of 92° C. for 1 min, 66° C. for 30 sec, 72° C. for 1 min, and a 10 min extension at 72° C. 8 μl of the PCR reaction was analyzed by gel electrophoresis for the presence of a GFP mRNA specific fragment of 131 nucleotides. This method detects GFP expression in a qualitative manner.

TABLE 1
Gene expression of GFP expression plasmid in HGPs 24 hours
following DNA delivery into skin. Positive gene expression is
defined as detectable signal in the rtPCR mRNA analysis.
N indicates the number of animals per group.

| | | | Donor | | Gene |
|---|---|---|---|---|---|
| | | DNA delivery | electrode | Electrical | Expression |
| Group | N | method | assembly | condition | (rtPCR) |
| 1 | 2 | Coated array | None | none | 2/2 negative |
| 2 | 4 | Coated array | 6 NA | EP | 4/4 positive |
| 3 | 4 | Coated array | Cathode gel | IO | 4/4 positive |
| 3A | 4 | Coated array + | Cathode gel | IO | 4/4 positive |
| | | Gel | | | |
| 4 | 4 | Coated array | Cathode gel | IO | 4/4 positive |
| | | | with array | | |
| 4A | 4 | Coated array + | Cathode gel | IO | 4/4 positive |
| | | Gel | with array | | |
| 5 | 4 | ID | None | none | 4/4 positive |
| 6 | 3 | None | None | none | 3/3 negative |

The rtPCR assay provides a sensitive but relatively non-quantitative method to determine gene expression on the mRNA level. As can be seen in Table 1, iontophoresis following DNA delivery by microprojection array produced gene expression in HGP skin, while delivery by microprojection alone did not result in detectable expression. This example demonstrates that intracellular delivery of DNA by iontophoresis after delivery to skin by microprojection array is feasible. Further, it demonstrates that electroporation may not be required for gene transfer.

### Example 2

When protein vaccines are delivered extra-cellularily, humoral responses are obtained, as the presentation of the antigen occurs via the class II MHC/HLA pathway. An additional cellular immune response is achieved only when protein vaccines are delivered into the cytosol (or when the antigen is produced intracellularly—as replicating vaccines or DNA vaccines). In this example, combination of transdermal polypeptide vaccine delivery by microprojection array technology using dry coated arrays or gel reservoirs with iontophoresis to assist intracellular delivery is studied. Immune responses to Hepatitis B virus surface antigen (HBsAg) protein are monitored. Nine treatment groups are evaluated:

Group 1: HBsAg protein-coated microprojection array (MA) delivery (5 min application time) without any iontophoresis or electroporation.

Group 2: HBsAg protein-coated microprojection array delivery (5 min application time) followed by 15 min iontophoresis after removal of the microprojection array.

Group 3: HBsAg protein-coated microprojection array delivery (5 min application time) followed by 15 min iontophoresis with the non-electroactive microprojection member left in the skin.

Group 4: Application of uncoated microprojection array followed by iontophoresis with HBsAg protein in gel reservoir after removal of the microprojection array. The gel reservoir is on the skin for 5 min prior to 15 min iontophoresis.

Group 4A: Application of uncoated microprojection array with HBsAg protein in gel reservoir after removal of the microprojection array, no iontophoresis. The gel reservoir is on the skin for 20 min.

Group 5: Application of uncoated microprojection array followed by iontophoresis with HBsAg protein in gel reservoir with the non-electroactive microprojection member left in the skin. The gel reservoir is on the skin for 5 min prior to 15 min iontophoresis.

Group 5A: Application of uncoated microprojection array with HBsAg protein in gel reservoir with the non-electroactive microprojection member left in the skin, no iontophoresis. The gel reservoir is on the skin for 20 min.

Group 6: HBsAg protein in gel reservoir is applied on skin for 5 min followed by 15 min iontophoresis.

Group 6A: HBsAg protein in gel reservoir is applied on skin for 20 min, no iontophoresis.

### Materials and Methods

Microprojection array coating: 30 μg HBsAg protein (Aldevron, Fargo, N.D.) per 2 cm2 1035 array, obtained by roller coater methodology using an aqueous formulation containing 20 mg/mL HBsAg protein, 20 mg/mL sucrose, 2 mg/mL HEC, and 2 mg/mL Tween 20.

For Groups 1, the system is comprised of an adhesive backing (diameter 2.6 cm) with a 2 cm² microprojection array adhered in the middle. For Groups 2, 4, and 4A, the system is comprised of an adhesive ring adhered to an adhesive backing ring (diameter 2.6 cm) with a 2 cm² microprojection array adhered in the middle. For Groups 3, 5 and 5A, the system is comprised of an adhesive backing ring (diameter 2.6 cm) with a 2 cm² microprojection array adhered in the middle. For Groups 2 and 3, the iontophoresis gels used for the anode assemblies are 350 μl of aqueous 0.15 M NaCl in 2% HEC (NATROSOL® 250 HHX PHARM, HERCULES Int. Lim., Netherlands, Mw 1890000, Mn 1050000). For Groups 4, 4A, 5, 5A, 6, and 6 A, the donor electrode assemblies consists of 350 μl 20 mM NaCl, 2% aqueous HEC gel proximate to the anode and a 175 μl, 20 mg/ml HbsAg protein, 25 mM His-Glu, 0.2% Tween 20, 1.5% aqueous HEC gel pH 5.2 proximate to the skin. The two gels are separated by an anionic exchange membrane (Sybron). For groups 2, 3, 4, 5, and 6 the iontophoresis gels used for the cathode assemblies are 350 μl of aqueous 0.15 M NaCl in 2% HEC.

Iontophoresis conditions: 4 mA for a total of 60 mA×minutes (15 minutes treatment time). Anode and cathode electrode assemblies are assembled immediately prior to application to skin by dispensing the indicated amounts of formulated HEC gels into the electrode assembly reservoirs. The distance (center) between anode and cathode is 3 cm. An iontophoresis power supply, DOMED phoresor II, Model No. PM100, is used.

HBsAg protein delivery to hairless guinea pig (HGP) skin: Coated microprojection arrays are applied to the flank of the anesthetized HGPs using an impact applicator. For Groups 1, 5 minutes after array application, the microprojection array adhered to the adhesive backing is removed. For Groups 2, 4, and 4A, 5 min after array application, the microprojection array adhered to the adhesive backing ring is removed, leaving the adhesive ring in contact with the skin and the donor electrode assembly is applied to the adhesive ring. For groups 3, 5, and 5A, 5 min after application, the donor electrode assembly is applied to the adhesive backing ring of the microprojection array. For groups 6 and 6A, the electrode assembly is applied on intact skin for 5 min prior to application of the electrical condition. The electrical condition (none or IO) is applied immediately following HBsAg protein delivery by microprojection array or gel, while all animals remain under anesthesia.

Humoral immune responses two weeks after one booster application using the same treatment conditions at week four are measured using the ABBOTT AUSAB EIA Diagnostic Kit and quantification panel. Antibody titers of higher than the protective level of 10 mIU/ml are marked as “positive” in Table 2.

Cellular responses are determined using a surrogate assay to predict CTL activity: spleen cells are harvested at the time of obtaining the sera for antibody titer determination and the number of gamma interferon producing CD8 cells—after depletion of CD4 positive cells by anti-CD4-coated Dynabeads (Dynal, N.Y.)— are determined by ELISPOT assay after a five day in vitro re-stimulation with the HBsAg protein. A “positive” response is scored when (i) mean number of cells in wells re-stimulated with HBsAg are significantly (P<0.05, student's t test) higher than in wells re-stimulated with ovalbumin (Ova), an irrelevant antigen (ii) net number of spot forming cells (SFCs) (SFCs in wells stimulated with HBsAg minus number of SFCs in wells stimulated with Ova) is 5 or larger, and (iii) the ratio of mean number of SFCs in HBsAg wells to mean number of SFCs in Ova wells is greater than 2.0.

TABLE 2
Treatment Table and Immune Responses

| | | HBsAg | Donor | | | |
|---|---|---|---|---|---|---|
| | | delivery | electrode | Electrical | Immune response | |
| Group | N | method | assembly | condition | humoral | cellular |
| 1 | 4 | Coated array | None | none | positive | negative |
| 2 | 4 | Coated array | Anode gel | IO | positive | positive |
| 3 | 4 | Coated array | Anode gel | IO | positive | positive |
| | | | with array | | | |
| 4 | 4 | Uncoated | Anode gel | IO | positive | positive |
| | | array, gel | | | | |
| 4A | 4 | Uncoated | Anode gel | none | positive | negative |
| | | array, gel | | | | |
| 5 | 4 | Uncoated | Anode gel | IO | positive | positive |
| | | array, gel | with array | | | |
| 5A | 4 | Uncoated | Anode gel | none | positive | negative |
| | | array, gel | with array | | | |
| 6 | 4 | Gel | Anode gel | IO | negative | negative |
| 6A | 4 | Gel | Anode gel | none | negative | negative |

This example demonstrates that iontophoresis in combination with protein delivery by microprojection array can result in humoral and cellular response to the polypeptide vaccine. Delivery by microprojection alone results in generation of a humoral response, while iontophoresis alone or passive permeation does not result in detectable immune response. This example demonstrates that intracellular delivery of protein antigens by iontophoresis after delivery to skin by microprojection array is feasible.

Without departing from the spirit and scope of this invention, one of ordinary skill can make various changes and modifications to the invention to adapt it to various usages and conditions. As such, these changes and modifications are properly, equitably, and intended to be, within the full range of equivalence of following claims.

1. A system for transdermally delivering a vaccine, comprising:
an agent formulation containing a vaccine, said formulation being adapted for transdermal delivery;
a non-electroactive microprojection member having a plurality of stratum corneum-piercing microprojections; and
an iontophoresis device having a donor electrode, a counter electrode, electric circuitry for supplying iontophoresis energy to the electrodes, and a donor electrode assembly including an electrolyte adapted and positioned to separate said donor electrode from said microprojection member.

2. The system of claim 1, wherein said agent formulation comprises a biocompatible coating disposed on said microprojection member, said agent formulation being formed from a coating formulation.

3. The system of claim 1, wherein said microprojection member has a microprojection density of at least approximately 10 microprojections/cm².

4. The system of claim 3, wherein said microprojection member has a microprojection density in the range of approximately 200-2000 microprojections/cm².

5. The system of claim 1, wherein said microprojection member comprises a material selected from the group consisting of stainless steel, titanium, and nickel titanium alloys.

6. The system of claim 1, wherein said microprojection member comprises a non-conductive material.

7. The system of claim 1, wherein said vaccine comprises a protein-based vaccine.

8. The system of claim 7, wherein supply of said iontophoresis energy to said electrodes provides in vivo intracellular delivery of said protein-based vaccine, whereby said delivery of said protein-based vaccine into skin-presenting cells leads to cellular loading of said protein-based vaccine epitopes onto class I MHC/HLA presentation molecules in addition to class II MHC/HLA presentation molecules in a subject.

9. The system of claim 8, wherein a cellular and humoral response is produced in said subject.

10. The system of claim 1, wherein said vaccine comprises a DNA vaccine.

11. The system of claim 10, wherein supply of said iontophoresis energy to said electrodes provides in vivo intracellular delivery of said DNA vaccine, whereby said delivery of said DNA vaccine leads to cellular expression of the vaccine antigen encoded by the DNA vaccine and loading of vaccine epitopes onto class I MHC/HLA presentation molecules in addition to class II MHC/HLA presentation molecules in a subject.

12. The system of claim 11, wherein a cellular and humoral response is produced in said subject.

13. The system of claim 11, wherein only a cellular response is produced in said subject.

14. The system of claim 1, wherein said vaccine is selected from the group consisting of viruses, weakened viruses, killed viruses, bacteria, weakened bacteria, killed bacteria, protein-based vaccines, polysaccharide-based vaccine, nucleic acid-based vaccines, proteins, polysaccharide conjugates, oligosaccharides, lipoproteins, Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), cytomegalo virus, hepatitis B virus, hepatitis C virus, human papillomavirus, rubella virus, varicella zoster, bordetella pertussis, clostridium tetani, corynebacterium diptheriae, group A streptococcus, legionella pneumophila, neisseria meningitdis, pseudomonas aeruginosa, streptococcus pneumoniae, treponema pallidum, vibrio cholerae, flu vaccines, lyme disease vaccines, rabies vaccines, measles vaccines, mumps vaccines, chicken pox vaccines, small pox vaccines, hepatitus vaccines, pertussis vaccines, diptheria vaccines, nucleic acids, single-stranded nucleic acids, double-stranded nucleic acids, supercoiled plasmid DNA, linear plasmid DNA, cosmids, bacterial artificial chromosomes (BACs), yeast artificial chromosomes (YACs), mammalian artificial chromosomes, RNA molecules, and mRNA.

15. The system of claim 1, wherein said formulation further comprises an immune response augmenting adjuvant selected from the group consisting of aluminum phosphate gel, aluminum hydroxide, alpha glucan, β-glucan, cholera toxin B subunit, CRL1005, ABA block polymer with mean values of x=8 and y=205, gamma inulin, linear (unbranched) β-D(2->1) polyfructofuranoxyl-α-D-glucose, Gerbu adjuvan, N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1H-imidazo[4,5-c]quinolin-4-amine, ImmTher™, N-acetylglucoaminyl-N-acetylmuramyl-L-Ala-D-isoGlu-L-Ala-glycerol dipalmitate, MTP-PE liposomes, C₅₉H₁₀₈N₆O₁₉PNa-3H₂O (MTP), Murametide, Nac-Mur-L-Ala-D-Gln-OCH₃, Pleuran, QS-21; S-28463,4-amino-a,a-dimethyl-1H-imidazo[4,5-c]quinoline-1-ethanol, sclavo peptide, VQGEESNDK.HCl (IL-1β 163-171 peptide), threonyl-MDP (Termurtide™), N-acetyl muramyl-L-threonyl-D-isoglutamine, interleukine 18 (IL-18), IL-2 IL-12, IL-15, IL-4, IL-10, DNA oligonucleotides, CpG containing oligonucleotides, gamma interferon, and NF kappa B regulatory signaling proteins.

16. The system of claim 2, wherein said coating formulation includes a surfactant.

17. The system of claim 16, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laurate, alkoxylated alcohols, and laureth-4.

18. The system of claim 2, wherein said coating formulation includes an amphiphilic polymer.

19. The system of claim 18, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

20. The system of claim 2, wherein said coating formulation includes a hydrophilic polymer.

21. The system of claim 20, wherein said hydrophilic polymer is selected from the group consisting of poly(vinyl alcohol), poly(ethylene oxide), poly(2-hydroxyethylmethacrylate), poly(n-vinyl pyrolidone), polyethylene glycol and mixtures thereof.

22. The system of claim 2, wherein said coating formulation includes a biocompatible carrier.

23. The system of claim 20, wherein said biocompatible polymer is selected from the group consisting of human albumin, bioengineered human albumin, polyglutamic acid, polyaspartic acid, polyhistidine, pentosan polysulfate, polyamino acids, sucrose, trehalose, melezitose, raffinose and stachyose.

24. The system of claim 2, wherein said coating formulation includes a stabilizing agent selected from the group consisting of a non-reducing sugar, a polysaccharide, a reducing sugar, and a DNase inhibitor.

25. The system of claim 2, wherein said coating formulation includes a vasoconstrictor.

26. The system of claim 25, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

27. The system of claim 2, wherein said coating formulation includes a pathway patency modulator.

28. The system of claim 27, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

29. The system of claim 2, wherein said coating formulation has a viscosity less than approximately 500 centipoise and greater than 3 centipoise.

30. The system of claim 2, wherein said coating has a thickness less than approximately 25 microns.

31. The system of claim 1, wherein said agent formulation comprises a hydrogel and wherein said system further includes an agent reservoir disposed adjacent said donor electrode, said agent reservoir being adapted to receive said hydrogel.

32. The system of claim 31, wherein said hydrogel comprises a macromolecular polymeric network.

33. The system of claim 32, wherein said macromolecular polymeric network is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

34. The system of claim 31, wherein said hydrogel includes a surfactant.

35. The system of claim 34, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laurate, alkoxylated alcohols, and laureth-4.

36. The system of claim 31, wherein said hydrogel includes an amphiphilic polymer.

37. The system of claim 36, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

38. The system of claim 31, wherein said hydrogel includes a pathway patency modulator.

39. The system of claim 38, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

40. The system of claim 31, wherein said hydrogel includes a vasoconstrictor.

41. The system of claim 40, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

42. The system of claim 1, wherein said microprojection member is an integral portion of said iontophoresis device.

43. The system of claim 1, further including an applicator having a contacting surface, wherein said microprojection member is releasably mounted on said applicator by a retainer and wherein said applicator, once activated, brings said contacting surface into contact with said microprojection member in such a manner that said microprojection member can strike a stratum corneum of a patient with a power of at least 0.05 joules per cm² of microprojection member in 10 milliseconds or less.

44. A method for transdermally delivering a vaccine to a subject, the method comprising the steps of:
providing an iontophoresis device having a donor electrode, a counter electrode, electric circuitry for supplying iontophoresis energy to said electrodes, a formulation including a vaccine, and a non-electroactive microprojection member having a plurality of stratum corneum-piercing microprojections;
placing said microprojection member in intimate contact with a patient's skin, wherein the microprojections pierce said patient's stratum corneum; and
supplying iontophoresis energy to said electrodes to transdermally deliver said vaccine.

45. The method of claim 44, further including the steps of:
providing an applicator having a contacting surface, wherein said microprojection member is releasably mounted on said applicator by a retainer; and
activating said applicator to bring said contacting surface into contact with said microprojection member in such a manner that said microprojection member strikes said stratum corneum.

46. The method of claim 45, wherein said step of activating said applicator causes said microprojection member to strike said stratum corneum with a power of at least 0.05 joules per cm² of microprojection member in 10 milliseconds or less.

47. The method of claim 46, further comprising the step of contacting said microprojection member with said iontophoresis device to supply said iontophoresis energy after activating said applicator.

48. The method of claim 44, further including the step of removing said microprojection member from said patient's stratum corneum before supplying said iontophoresis energy.

49. The method of claim 44, wherein said microprojection member is an integral portion of said iontophoresis device.

50. The method of claim 44, wherein said step of supplying iontophoresis energy to said electrodes comprises applying a current in the range of approximately 50 μA-20 mA over a time period in the range of approximately 1.0 min to 1 day.

51. The method of claim 44, wherein said step of supplying iontophoresis energy to said electrodes comprises applying a voltage in the range of approximately 0.5 V -20 V over a time period in the range of approximately 1.0 min to 1 day.

52. The method of claim 44, wherein said vaccine comprises a protein-based vaccine.

53. The method of claim 52, wherein said supply of said iontophoresis energy to said electrodes provides in vivo intracellular delivery of said protein-based vaccine, whereby said delivery of said protein-based vaccine into skin-presenting cells leads to cellular loading of said protein-based vaccine epitopes onto class I MHC/HLA presentation molecules in addition to class II MHC/HLA presentation molecules in a subject.

54. The method of claim 53, wherein a cellular and humoral response is produced in said subject.

55. The method of claim 44, wherein said vaccine comprises a DNA vaccine.

56. The method of claim 55, wherein said supply of said iontophoresis energy to said electrodes provides in vivo intracellular delivery of said DNA vaccine, whereby said delivery of said DNA vaccine leads to cellular expression of the vaccine antigen encoded by the DNA vaccine and loading of vaccine epitopes onto class I MHC/HLA presentation molecules in addition to class II MHC/HLA presentation molecules in a subject.

57. The method of claim 56, wherein a cellular and humoral response is produced in said subject.

58. The method of claim 56, wherein a cellular response is produced in said subject.
